(19) United States
(12) Patent Application Publication
(10) Pub. No.: US 2024/0142422 A1
(43) Pub. Date: May 2, 2024

(54) Marinized Distributed Acoustic Sensing System

(71) Applicant: Halliburton Energy Services, Inc., Houston, TX (US)

(72) Inventors: Glenn Wilson, Houston, TX (US); Andreas Ellmauthaler, Houston, TX (US); John Laureto Maida, Houston, TX (US)

(73) Assignee: Halliburton Energy Services, Inc., Houston, TX (US)

(21) Appl. No.: 17/977,987

(22) Filed: Oct. 31, 2022

(51) Int. Cl. G01N 33/00; G01K 11/322; G01N 21/01

(52) U.S. Cl. CPC G01N 33/0031; G01K 11/322; G01N 21/01; G01N 33/0016; G01N 33/0073; G01N 2201/0833; G01N 2201/084

(57) ABSTRACT

A method and system for a fiber optic sensing (FOS) system. The system may include an interrogator that is marinized to be disposed on a sea floor, a fiber optic cable optically connected to the interrogator, and one or more downhole sensing fibers optically connected to the fiber optic cable. The method may include disposing the interrogator on the sea floor and connecting the interrogator to a fiber optic cable. The method may further include connecting one or more downhole sensing fibers to the fiber optic cable and taking one or more measurements using the interrogator.

### BACKGROUND

Boreholes drilled into subterranean formations may enable recovery of desirable fluids (e.g., hydrocarbons), or geological storage of other fluids (e.g., carbon dioxide), using a number of different techniques. A number of fiber optic sensing (FOS) systems and techniques may be employed in subterranean operations to characterize and monitor borehole and/or formation properties. For example, Distributed Temperature Sensing (DTS) and Distributed Acoustic Sensing (DAS) along with a fiber optic system may be utilized together to determine borehole and/or formation properties including production profiling, solids production, injection profiling, flow assurance, vertical seismic profiling, well integrity, geological integrity, and leak detection. Distributed fiber optic sensing is a cost-effective method of obtaining real-time, high-resolution, highly accurate temperature and/or strain (static or dynamic, including acoustic) and/or pressure data along the entire wellbore. Discrete (or point) fiber optic sensing, e.g., by using fiber Bragg gratings (FBGs), is an alternative cost-effective method of obtaining real-time, high resolution, highly accurate temperature and/or strain data at discrete locations along the wellbore. Moreover, FBGs and the downhole cable may be integrated with transducers capable of inducing temperature and/or strain upon at least one FBG, thus providing an optically proportional measure of transduction, e.g., for sensing pressure, voltage, current, or chemical concentration. Additionally, fiber optic sensing may eliminate downhole electronic complexity by shifting all electrical and electro-optical systems to the surface within the interrogator(s). Fiber optic cables may be permanently deployed downhole in a wellbore via single- or dual-trip completion strings, behind casing, on tubing, or in pumped down installations; or temporally via coiled tubing, wireline, slickline, or disposable cables.

Distributed fiber optic sensing may be enabled by continuously sensing along the length of the optical fiber, and effectively assigning discrete measurements to a position or set of positions along the length of the fiber via optical time-domain reflectometry (OTDR). That is, by knowing the velocity of light in fiber, and by measuring the time it takes the backscattered light to return to the detector inside the interrogator, it is possible to assign a measurement and distance along the fiber. In alternative embodiments, functionally equivalent distributed fiber optic sensing data may be acquired via optical frequency-domain reflectometry (OFDR) techniques.

DAS, DTS, and FBG sensing has been practiced for monitoring downhole sensing fibers in dry Christmas tree (or dry-tree) wells to enable interventionless, time-lapse temperature, acoustic, and pressure monitoring borehole and/or formation properties including production profiling, solids production, injection profiling, flow assurance, vertical seismic profiling, well integrity, geological integrity, and leak detection. For installation in dry-tree wells, multiple sensing fibers are typically integrated in a tubing encapsulated fiber (TEF) cable. This enables, for example, a DAS system to preferentially sense a single-mode downhole sensing fiber, and a DTS system to preferentially sense a multi-mode downhole sensing fiber; such that the DAS and DTS systems are operated simultaneously but are not simultaneously sensing the same downhole sensing fiber. Typically, the interrogators are adjacent to, or a short distance, from the well head outlet on the dry Christmas tree.

For downhole sensing fibers installed in subsea wells, interrogator system(s) may be deployed on the topside facility, and to sense the downhole sensing fiber through optical distribution in the subsea infrastructure. However, such a subsea well sensing operation then requires optical engineering solutions to compensate for insertion losses accumulated through long (˜5 to 100+km) lengths of subsea transmission fiber between the topside facility and subsea tree (e.g., static umbilical lines, dynamic umbilical lines, jumper cables, optical flying leads), up to 10 km of downhole sensing fiber, and multiple wet- and dry-mate optical connectors, splices, and an optical feedthrough systems (OFS) in the subsea Christmas tree (XT).

### BRIEF DESCRIPTION OF THE DRAWINGS

These drawings illustrate certain aspects of some of the embodiments of the present disclosure, and should not be used to limit or define the disclosure:

FIGS. 1A-1D illustrate examples of a well measurement system in a subsea environment;

FIGS. 2A-2C illustrates examples of a downhole fiber deployed in a wellbore;

FIG. 3 illustrates an optical distribution unit;

FIG. 4 illustrates an umbilical termination assembly;

FIG. 5 illustrates an optical flying lead;

FIG. 6A illustrates an optical feedthrough system;

FIG. 6B illustrates a cutaway of at least a part of subsea tree;

FIG. 7 illustrates an example of a FOS system; and

FIG. 8 illustrates an example of a FOS system with a marinized interrogator.

### DETAILED DESCRIPTION

The present disclosure relates generally to a system and method for fiber optic sensing by disposing an interrogator directly on the sea floor to reduce fiber optic length. Fiber optic sensing may comprise Fiber Bragg Gratings (FBGs), Distributed Acoustic Sensing (DAS), Distributed Temperature Sensing (DTS), Distributed Strain Sensing (DSS), Distributed Chemical Sensing (DCS), Distributed Magnetomotive Force Sensing (DMS), Distributed Electromotive Force Sensing (DES), and Distributed Brillouin-Frequency Sensing (DBFS), the latter which may be used in the extraction of distributed strain, temperature, or pressure or a combination thereof. It should be noted that any, or any combination of all systems and methods described above are generally referred to as a Fiber Optic Sensing (FOS) system. The sensing region of interest is typically the downhole sensing fiber (i.e., the in-well and reservoir sections), and not the transmission fibers (i.e., OFLs, jumpers, and static and/or dynamic umbilical lines).

To prevent a reduction in FOS signal-to-noise (SNR) and signal quality and fidelity, the FOS system described below may increase the returned signal strength with given pulse power for emitted light, decrease the noise floor of the receiving optics to detect weaker power pulses, maintain the pulse power as high as possible as it propagates along the transmission fiber(s), increase the number of light pulses that may be launched into the downhole sensing fiber(s) per second, and/or increase the maximum pulse power that may be used for given fiber length.

FOS systems utilize one or more downhole sensing fibers integrated in fiber optic cables (or tubing encapsulated fibers, TEFs). One or more electrical conductors may be integrated in the TEF so as to provide electrical power and/or telemetry to a downhole device, e.g., a pressure gauge. Downhole sensing fibers may be at least one single-mode fiber (SMF), at least one multi-mode fiber (MMF), or a combination of at least one SMF and at least one MMF. Each of the at least one SMF or MMF may be treated with a coating to prevent undesirable effects, e.g., hermetically sealed in carbon to delay hydrogen degradation. Each of at least one SMF or MMF may be treated with a coating to generate desirable effects, e.g., induced strain via improved strain transduction, a chemical reaction, or exposure to an electromotive or magnetomotive force. At least one SMF may further be enhanced (or engineered) to yield a higher-than-Rayleigh scattering coefficient so as to increase the DAS signal to noise ratio (SNR) by 10 dB to 20 dB. Such enhanced backscatter fibers (EBF) may comprise of either weak, distributed gratings, or discrete gratings in a SMF. The EBF may be fabricated with a narrow enhanced backscatter bandwidth, such that a DAS system may be sensitive to the enhanced backscatter, but at least one other FOS system does not exhibit any appreciable sensitivity to the enhanced backscatter than it would if sensing a standard (or non-enhanced) SMF. The EBF may be fabricated with a broad enhanced bandwidth, such that a DAS system and at least one other FOS system may exhibit sensitivity to the enhanced backscatter.

Fiber optic cables may be permanently deployed in a subsea well via single- or dual-trip completions. Fiber optic cables may comprise one of at least one optical fiber encapsulated in a hydrogen-scavenging gel-filled stainless steel tube and may further be encapsulated in a metallic (e.g., Inconel® alloy 825) armor. A hydrogen delay barrier may be located between the stainless-steel tube and the armor, e.g., a metallurgical hydrogen delay barrier such as aluminum may be extruded upon the stainless-steel tube before encapsulation in the metallic armor. The fiber optic cables may be further encapsulated in a thermoplastic encapsulation.

FOS systems utilize transmission fibers integrated in the subsea infrastructure fiber optic cables to provide optical continuity between the interrogator(s) located at the topside facility and downhole sensing fiber(s) in the subsea well. The transmission fibers may be integrated within OFLs, jumpers, and static and/or dynamic umbilical lines, and optically coupled via splices, wet-mate connectors, and/or dry-mate connectors. Transmission fibers may be either SMF or MMF. In some embodiments, the transmission fibers may be low-loss (LL) or ultra-low loss (ULL) SMFs that have lower optical attenuation and higher power handling capability before non-linearity so as to enable high gain, co- or counter-propagating distributed Raman amplification. For example, pure silica core SMF, such as Corning® SMF-28® ULL SMF, typically exhibit 0.15 to 0.17 dB/km optical attenuation at 1550 nm wavelengths.

FOS systems may employ distributed fiber optic sensing, which is a cost-effective method of obtaining real-time, high-resolution, highly accurate temperature, strain, and acoustic/vibration data along the entire downhole fiber, while simultaneously eliminating downhole electronic complexity by shifting all electro-optical system complexity to the interrogator(s) located at the topside facility. Example of distributed fiber optic sensing comprise distributed acoustic sensing (DAS), also referred to as distributed vibration sensing (DVS), which preferentially operates with SMF; distributed Brillouin-frequency sensing for distributed temperature and/or strain sensing and/or pressure sensing (DTS/DSS/DPS) preferentially operates with SMF; and Raman DTS which preferentially operates with MMF. Other distributed fiber optic sensing may comprise distributed chemical sensing (DCS), distributed electromotive force sensing (DES), and distributed magnetomotive force sensing (DMS).

Distributed fiber optic sensing may operate by continuously sensing along the length of the downhole sensing fiber, and effectively assigning discrete measurements to a position along the length of the fiber via optical time-domain reflectometry (OTDR). That is, by knowing the velocity of light in fiber, and by measuring the time it takes the backscattered light to return to the detector inside the interrogator, it is possible to assign a distance along the fiber. In alternative embodiments, functionally equivalent distributed fiber optic sensing data may be acquired via optical frequency-domain reflectometry (OFDR) techniques.

Discrete, or point, fiber optic sensing is an alternative cost-effective method of obtaining real-time, high-resolution, highly accurate temperature and/or strain (acoustic) data at discrete locations/points along the entire wellbore, while simultaneously eliminating downhole electronic complexity by shifting all electro-optical complexity to the interrogator(s) located at the topside facility. Point sensors may comprise one or more fiber Bragg gratings (FBGs), where the optical waveguide containing the FBG may be modified by a sensor assembly which efficiently transduces a measurement to temperature and/or strain upon at least one FBG. An example of such a sensor assembly is a pressure and temperature gauge, a chemical sensor, and a voltage sensor. FBGs may operate with either SMF or MMF.

The subsea well's downhole sensing fiber connects to the subsea optical distribution system via an optical feedthrough system (OFS) in the subsea Christmas tree (XT) and tubing hanger. The XT may be either a vertical (VXT) or a horizontal XT (HXT) design, or any hybrid or simplified solution where to hang off the downhole completions. The methods and systems described below are agnostic to the use of VXTs or HXTs. In the following description, VXT, HXT, subsea Christmas tree, wet Christmas tree, wet-tree, and subsea tree are all synonymous. The OFS provides optical continuity from transmission fibers in the subsea optical distribution system to the downhole sensing fiber via an assembly of wet- and dry-mate optical connectors and/or splices. When the XT is landed on the tubing hanger, the OFS enables at least one fiber to be optically continuous between the XT's ROV panel and the tubing hanger. Current and future OFS products from TE Connectivity and Teledyne enable at most one, three, or six fibers to be fed through the XT. Fibers may be SMF, MMF, or any combination of SMF and MMF.

From a downhole monitoring system consideration, multiple downhole fibers may increase data acquisition opportunities while simplifying overall downhole monitoring system complexity. For example, one SMF may be used for acquiring DAS and/or DTS, and two SMFs may each or both be used for FBG sensing arrays of pressure and temperature gauges. For intelligent completions, this may potentially eliminate the necessity of electric pressure and temperature gauge arrays, and thus simplify subsea control and power distribution systems. The challenge is that having multiple downhole sensing fibers with their necessity for optical continuity back to the interrogators located at the topside facility, which could place significant complexity, burden, and cost on the subsea optical distribution system. On a per-well basis, the systems and methods described below may maximize the number of downhole sensing fibers while minimizing the number of subsea transmission fibers needed for their continuity from XT to the topside facility.

The subsea optical distribution system provides optical continuity from the downhole sensing fiber to the interrogator located at the topside facility. The optical distribution system may be stand-alone (separated) or integrated with other (e.g., electric and/or hydraulic) utilities of the subsea production system (SPS). This may involve multiple optical flying leads (OFLs), jumper cables, static umbilical lines, dynamic umbilical lines, subsea umbilical termination assemblies (SUTAs), topside umbilical termination assemblies (TUTAs), surface cables between the TUTAs and interrogator(s), optical distribution units (ODUs), and optical distribution through drill centers, manifold centers, or other subsea equipment.

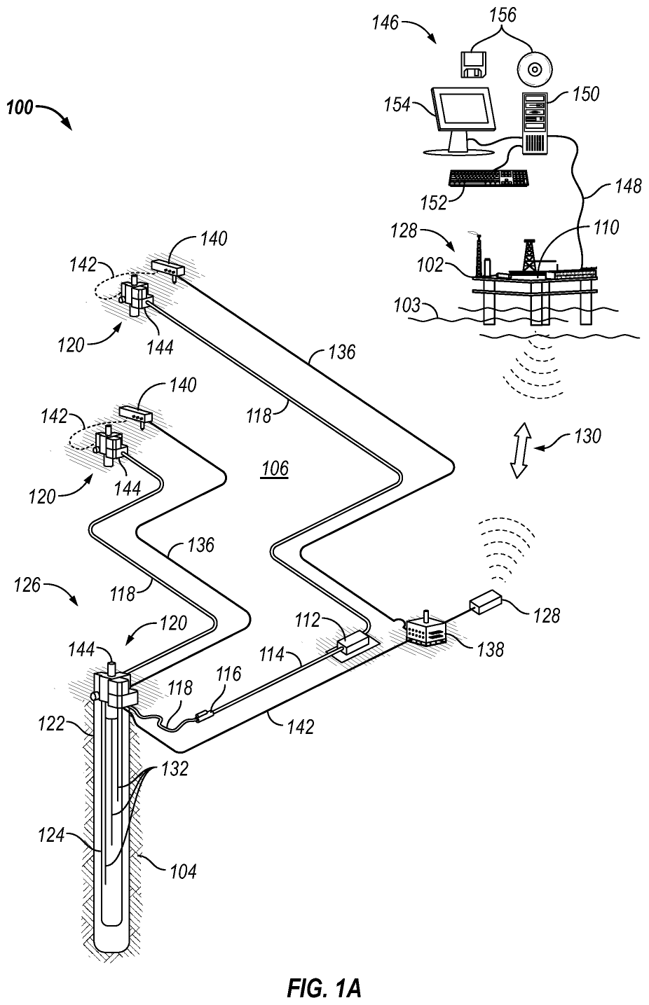

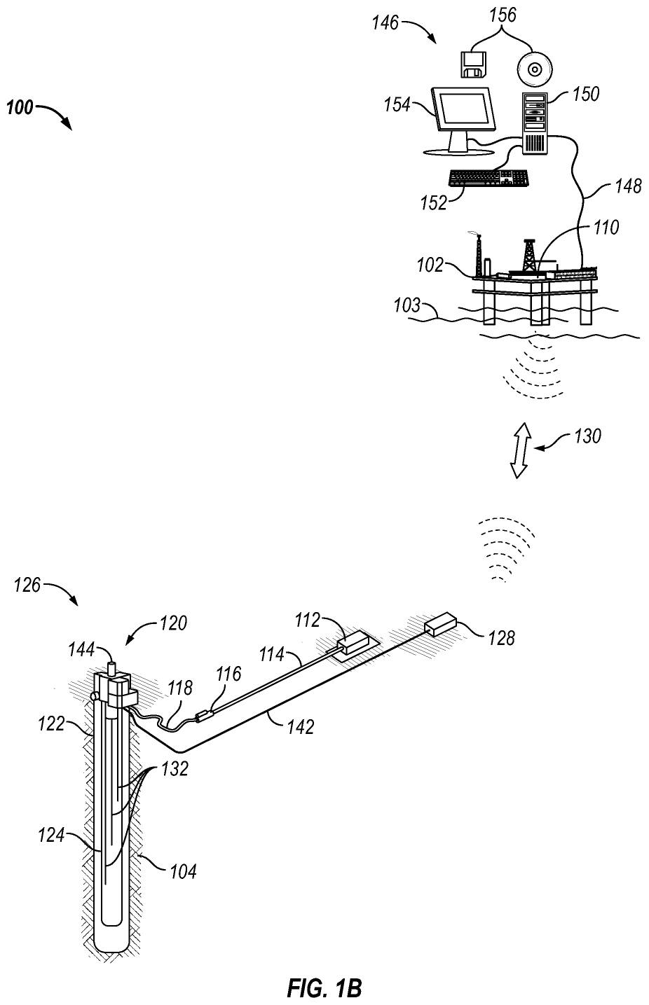

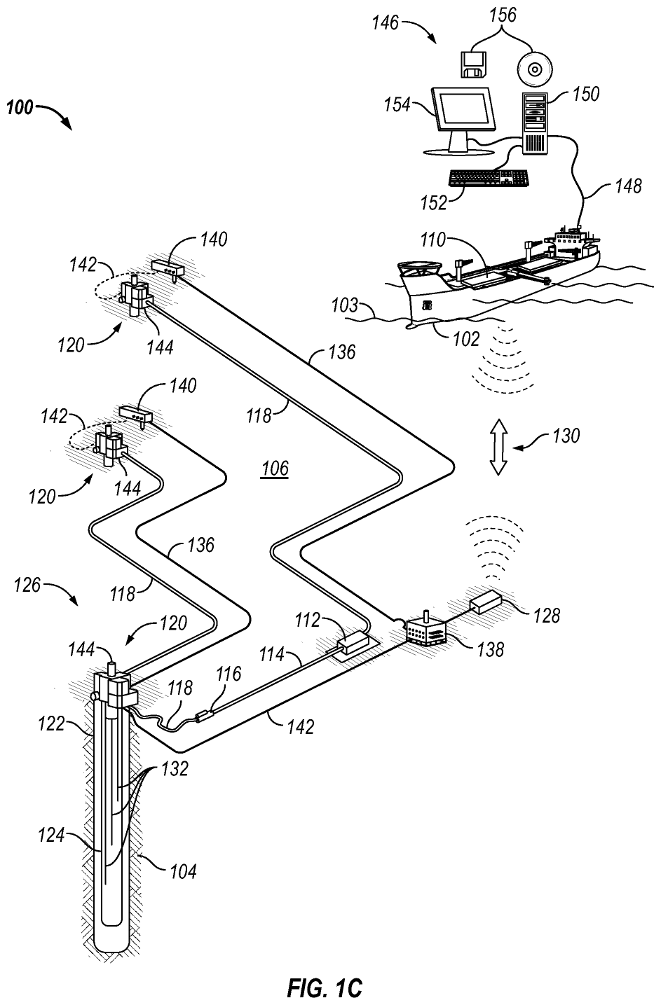

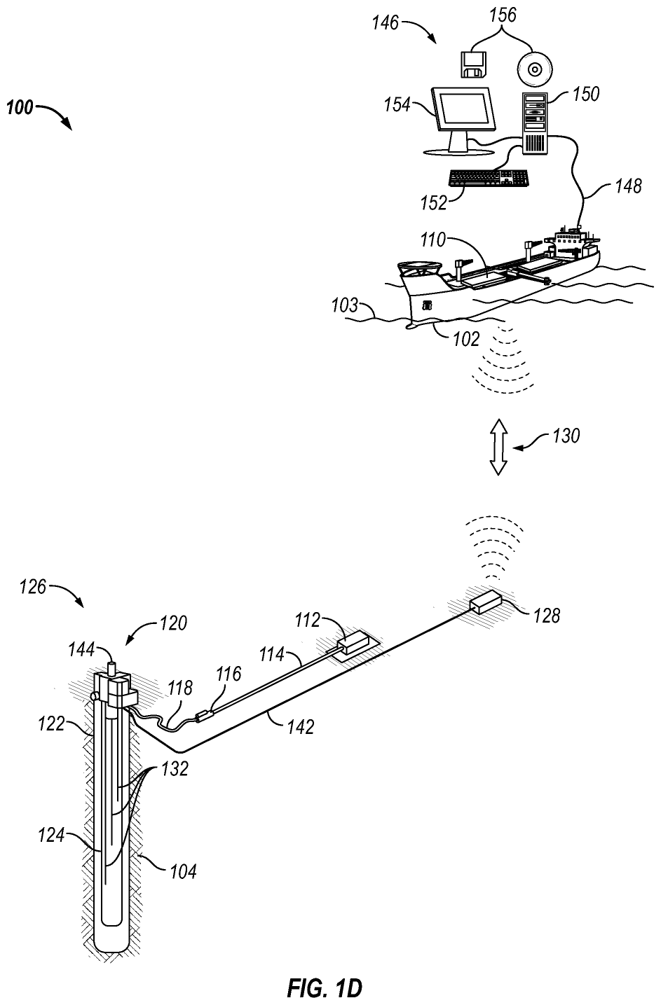

FIGS. 1A-1D illustrate an example of a well system 100 that may employ the principles of the present disclosure. More particularly, well system 100 may comprise a floating vessel 102 centered over a subterranean hydrocarbon bearing formation 104 located below a sea floor 106. As illustrated, floating vessel 102 is depicted as an offshore, semi-submersible oil and gas drilling platform, but could alternatively comprise any other type of floating vessel such as, but not limited to, a drill ship, a pipe-laying ship, a tension-leg platforms (TLPs), a spar platform, a production platform, a floating production, storage, and offloading (FPSO) vessel, a floating production unit (FPU), and/or the like. Additionally, and without loss of generality, the methods and systems described below may also be utilized for subsea tie-backs to a fixed offshore platform, an onshore facility, or a facility on an artificial island. Moreover, the systems and methods of the present disclosure are applicable to onshore reservoirs and related facilities. For this disclosure, well system 100 may be performing long term measurement operations. During long term measurement operations, subsea conduit or risers are not present to attach to a deck 110 of floating vessel 102 to a production manifold 112. As there are not current production operations, riser and subsea conduit may not be utilized. As illustrated, static pipe 114 may run from production manifold 112 to a pipeline end termination 116. Flexible pipe 118 may attach a subsea tree 120 (e.g., subsea Christmas tree (XT)) to pipeline end termination 116. In examples, flexible pipe 118 may traverse from production manifold 112 and connect directly to subsea tree 120. Additionally, flexible pipe 118 may connect one subsea tree 120 to another subsea tree 120, effectively tying one or more subsea trees 120 together and allow for a single flexible pipe 118 to connect one or more subsea trees 120 to a single production manifold 112.

For measurement operations, a Fiber Optic Sensing (FOS) system 126 may be employed and disposed on sea floor 106. FOS 126 system utilizes distributed and/or discrete fiber optic sensing as a cost-effective method of obtaining, high-resolution, highly accurate physical measurements, such as but not limited to temperature, strain, and acoustic measurements along the entire wellbore, while simultaneously eliminating downhole electronic complexity by shifting all electro-optical complexity to the interrogator (IU), also called an interrogator. FOS system 126 may comprise an interrogator 128, a static umbilical line 136 or optical flying lead 142, and at least one downhole sensing fiber 132. Interrogator 128 may utilize optical backscattering phenomena based on Brillouin, Raman, and/or Rayleigh scattering in optical fibers to measure distributed temperature, static strain, and dynamic strain (acoustics & vibration) or chemical compositions/concentrations along the wellbore. Similarly, interrogator 128 may make use of point or quasi-distributed Fiber Bragg Grating resonances or other optical interferometric cavities (e.g., Fabry-Perot, Michelson, Mach-Zehnder, Sagnac configurations) along downhole sensing fiber 132 to measure point or quasi-distributed temperature, pressure, acoustics/vibration, chemical specie/concentrations, and/or electromagnetic/magnetic fields of interest. In examples, interrogator 128 may sense the effects of tubular corrosion within wellbore 122, via hydrogen generation, for tubular and casing health prediction monitoring. Additionally, downhole sensing fiber 132 may be able to detect injected CO2 plume extent/concentration. As illustrated, interrogator 128 may communicate to floating vessel 102 using wireless communication 130. Wireless communication 130 may comprise underwater ultrasonics and/or underwater laser based optical high speed telemetry technologies.

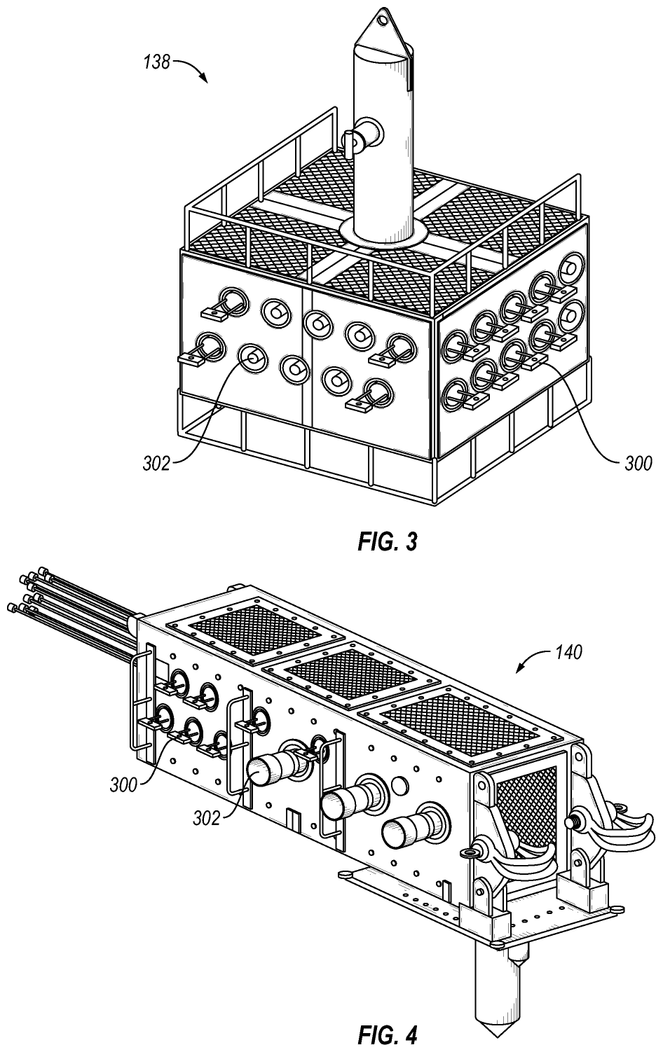

FIG. 3 illustrates an optical distribution unit 138. As illustrated, optical distribution unit 138 may be constructed to withstand pressures, temperatures, and a subsea environment in which optical distribution unit 138 may operate and function. During operations, a remotely operated vehicle (ROV) (not illustrated) may be deployed from vessel 102 or another vessel with optical distribution unit 138. The ROV may place optical distribution unit 138 in a previously designated area on sea floor 106. Once deployed, optical distribution unit 138 may act as a terminal to which interrogator 128 may attach (e.g., referring to FIGS. 1A and 1B). One or more ROVs may be utilized to attach dynamic umbilical line 134 and static umbilical line 136 to optical distribution unit 138. Additionally, this procedure, in some operations, may be performed at the surface on vessel 102. In examples, one or more dynamic umbilical lines 134 may attach to one or more input connectors 300. This may allow for one or more static umbilical lines 136 to connect to one or more output connectors 302. Thus, one or more static umbilical lines 136 may allow for a single vessel 102 to service one or more subsea trees 120 that are connected to optical distribution unit 138. To reach subsea trees 120, one or more static umbilical lines 136 traverse to one or more umbilical termination assemblies 140. Additionally, in examples, a flying optical lead 142 (discussed below) may be utilized to connect optical distribution unit 138 to one or more subsea trees 120.

FIG. 4 illustrates an umbilical termination assembly 140. As illustrated, umbilical termination assembly 140 may be constructed to withstand pressures, temperatures, and a subsea environment in which umbilical termination assembly 140 may operate and function. During operations, one or more ROVs (not illustrated) may be deployed from vessel 102 or another vessel with umbilical termination assembly 140. The ROV may place umbilical termination assembly 140 in a previously designated area on sea floor 106. Once deployed, umbilical termination assembly 140 may act as a terminal in which static umbilical line 136 attaches to from optical distribution unit 138 (e.g., referring to FIGS. 1A and 1B). One or more ROVs may be utilized to attach static umbilical line 136 to umbilical termination assembly 140. Additionally, this procedure, in some operations, may be performed at the surface on vessel 102. In examples, one or more dynamic umbilical lines 134 may attach to one or more input connectors 300. From umbilical termination assembly 140, an optical flying lead 142 may connect umbilical termination assembly 140 at one or more output connectors 302 to an optical feedthrough system 144 that is disposed in or is at least a part of subsea tree 120 (e.g., referring to FIGS. 1A and 1B).

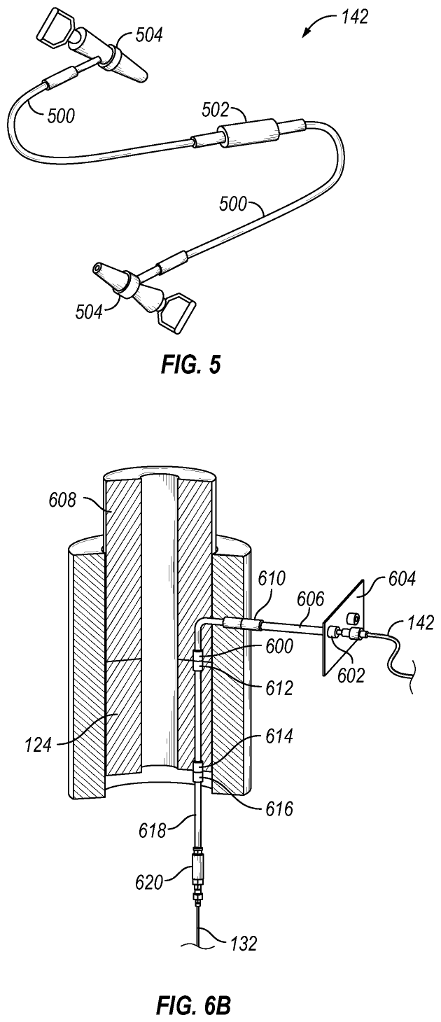

FIG. 5 illustrates an optical flying lead. An optical flying lead 142 is a flexible connection that may attach optical distribution unit 138 or umbilical termination assembly 140 or any other suitable location in the optical distribution system to optical feedthrough system 144. As illustrated, optical flying lead 142 comprises a flexible hose 500 terminated at both ends with optical wet-mate connectors 504. Flexible hose 500 comprises one or more optical fibers that provide optical continuity between the two optical wet-mate connectors 504. Flexible hose 500 may be filled with fluid for pressure balancing in subsea environments. Additionally, an integrated compartment 502 may be disposed at any distance along the flexible hose 500. Integrated compartment 502 may comprise any number of optical devices, which is discussed in detail below. Integrated compartment 502 may be rated as a one atmosphere (1 atm) pressure cannister qualified for deployment in subsea environments and may contain a nitrogen-purged atmospheric environment. Each optical wet-mate connection 504 is configured to allow for an ROV to attach optical flying lead 142 to optical feedthrough system 144 and optical distribution unit 138 or umbilical termination assembly 140 or any other suitable location in the optical distribution system, as is readily understood to those of ordinary skill in the art.

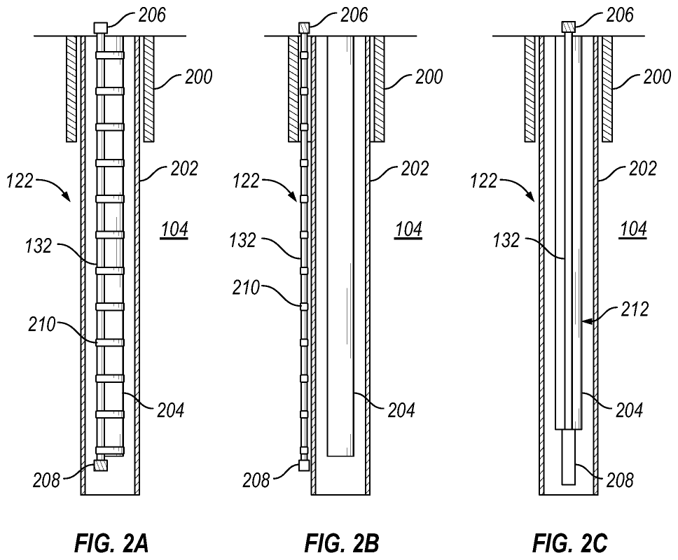

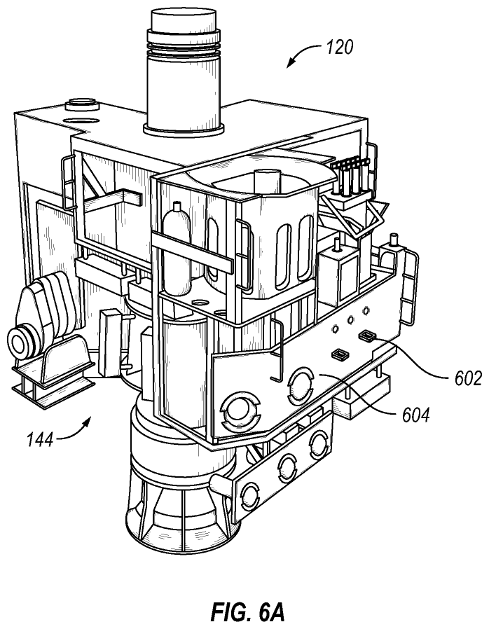

FIG. 6A illustrates a subsea tree 120 with optical feedthrough system 144. As illustrated, subsea tree 120 with optical feedthrough system 144 may be constructed to withstand pressures, temperatures, and a subsea environment in which subsea tree 120 and optical feedthrough system 144 may operate and function. During manufacturing of subsea tree 120, optical feedthrough system 144 may be integrated into subsea tree 120 and tubing hanger assemblies. Subsea tree 120 and tubing hanger assemblies each contain an optical wet-mate receptacle 600 (e.g., referring to FIG. 6B) that may be optically coupled when subsea tree 120 and tubing hangers are operationally deployed. During installation operations, the tubing hanger assembly is coupled to the upper completion of wellbore 122 with optical continuity to downhole sensing fiber 132 (e.g., referring to FIGS. 1A and 1B), and landed into wellbore 122 on sea floor 106 (e.g., referring to FIGS. 1A and 2B). Subsea tree 120 is then landed upon the tubing hanger such that subsea tree 120 and tubing hanger are optically coupled via the mated optical wet-mate receptacle 600. One or more ROVs may be utilized to attach optical flying lead 142 (e.g., referring to FIGS. 1A and 1B) to optical wet-mate receptacle 602 located on the ROV panel 604 of subsea tree 120 and optical feedthrough system 144 as well as optical distribution unit 138 or umbilical termination assembly 140. In examples, one or more static umbilical lines 136 may attach directly to subsea trees 120 without optical flying lead 142. Subsea tree 120 and optical feedthrough system 144 may allow for optical flying lead 142 and/or one or more static umbilical lines 136 to connect to one or more downhole sensing fibers 132.

FIG. 6B illustrates optical feedthrough system 144 formed when subsea tree 120 (e.g., referring to FIG. 6A) has been landed upon a tubing hanger. In examples, optical flying lead 142 may attach optical wet-mate receptacle 602 located on ROV panel 604 of subsea tree 120 (e.g., referring to FIGS. 6A), which is connected to a pressure-compensated flexible hose 606 that terminates with a an optical dry-mate connection 610 at subsea tree block 608. Optical dry-mate connection 600 is connected to the subsea tree's optical wet-mate receptacle 600. During installation operations, subsea tree 120 is landed upon the tubing hanger such that subsea tree's optical wet-mate receptacle 600 optically connects to tubing hanger's optical wet-mate receptacle 612. In some embodiments, the tubing hanger's optical wet-mate receptacle 612 is connected to an optical dry-mate receptacle 614 at the base of the tubing hanger, and optically connected to a pigtail 618 with optical dry-mate receptacle 616. Pigtail 618 is connected to downhole sensing fiber 132 via a splice assembly 620 in the upper completion. In other embodiments, tubing hanger's optical wet-mate receptacle 612 is optically connected to downhole sensing fiber 132 via a splice assembly 620 in the upper completion. Although not illustrated, one or more downhole sensing fibers 132 may be disposed in a fiber optic cable that is optically connected to tubing hanger's optical wet-mate receptacle 612. In examples, an integrated compartment 502 may be installed along flexible hose 606 between subsea tree's ROV panel 604 and the optical dry-mate connection 600 at subsea tree block 608. This integrated compartment may comprise any number of optical devices, which is discussed in detail below. Integrated compartment 502 may be a one atmosphere (1 atm) pressure cannister rated for deployment in subsea environments and may contain a nitrogen-purged atmospheric environment. As illustrated, and discussed below in further detail, optical feedthrough system 144 allows for optical coupling between optical flying lead 142 and one or more downhole sensing fibers 132 through a single connection. As will be discussed in more detail below, downhole sensing fibers 132 may allow for downhole measurements to be taken within wellbore 122 utilizing principles and function associated with FOS 126.

Referring back to FIGS. 1A and 1B, wellbore 122 extends through the various earth strata toward the subterranean hydrocarbon bearing formation 104 and tubular 124 may be extended within wellbore 122. Even though FIGS. 1A-1D depict a vertical wellbore 122, it should be understood by those skilled in the art that the methods and systems described are equally well suited for use in horizontal or deviated wellbores. During drilling operations, a drill string may comprise a bottom hole assembly (BHA) that comprises a drill bit and a downhole drilling motor, also referred to as a positive displacement motor (“PDM”) or “mud motor.” During production operations, the completion system represented by tubular 124 may comprise one or more downhole sensing fibers 132 of a FOS system 126.

Downhole sensing fiber 132 may be permanently deployed in a wellbore via single- or dual-trip completion systems, behind casing, on tubing, or in pumped down installations. FIGS. 2A-2C illustrate examples of different types of deployment of downhole sensing fiber 132 in wellbore 122 (e.g., referring to FIGS. 1A and 1B). As illustrated in FIG. 2A, wellbore 122 deployed in formation 104 may comprise surface casing 200 in which production casing 202 may be deployed. Additionally, production tubing 204 may be deployed within production casing 202. In this example, downhole sensing fiber 132 may be permanently deployed in a completion system. In examples, downhole sensing fiber 132 is attached to the outside of production tubing 204 by one or more cross-coupling protectors 210. Without limitation, cross-coupling protectors 210 may be evenly spaced and may be disposed on every other joint of production tubing 204. Further illustrated, downhole sensing fiber 132 may be coupled to a fiber connection 206. Without limitation, fiber connection 206 may attach downhole sensing fiber 132 to optical feedthrough system 144, and/or interrogator 128 (e.g., referring to FIGS. 1A and 1B) in the manner, systems, and/or methods described above. In examples, downhole sensing fiber 132 may further be optically connected to interrogator 128 through optical flying lead 142 (e.g., referring to FIGS. 1A and 1B). Fiber connection 206 may operate as an optical feedthrough system 144 (itself comprising a series of wet- and dry-mate optical connectors and splices) in the wellhead that optically connects downhole sensing fiber 132 from the tubing hanger to interrogator 128 on the subsea tree's ROV panel 604 (e.g., referring to FIGS. 6A and 6B). Interrogator 128 may optically connect to an optical flying lead 142 and may further comprise an optical distribution system(s) 138, umbilical termination unit(s) 140, and transmission fibers encapsulated in flying optical leads 142, flow lines, rigid risers, flexible risers, and/or one or more static and/or dynamic umbilical lines. This may allow for interrogator 128 to connect and disconnect from downhole sensing fiber 132 while preserving optical continuity between optical distribution unit 138 and the downhole sensing fiber 132.

FIG. 2B illustrates an example of permanent deployment of downhole sensing fiber 132. As illustrated in wellbore 122 deployed in formation 104 may comprise surface casing 200 in which production casing 202 may be deployed. Additionally, production tubing 204 may be deployed within production casing 202. In examples, downhole sensing fiber 132 is attached to the outside of production casing 202 by one or more cross-coupling protectors 210. Without limitation, cross-coupling protectors 210 may be evenly spaced and may be disposed on every other joint of production tubing 204. downhole sensing fiber 132

FIG. 2C illustrates an example of a pump-down fiber operation in which downhole sensing fiber 132 may be deployed either permanently or temporarily. As illustrated in FIG. 2C, wellbore 122 deployed in formation 104 may comprise surface casing 200 in which production casing 202 may be deployed. Additionally, capillary tubing 212 may be deployed within production casing 202. In this example, downhole sensing fiber 132 may be permanently or temporarily deployed via a pumping operation into the capillary tube.

Referring back to FIGS. 1A and 1B, interrogator 128 may be connected to an information handling system 146 through connection 148, which may be wired and/or wireless. It should be noted that both information handling system 146 and interrogator 128 are disposed on floating vessel 102. Both systems and methods of the present disclosure may be implemented, at least in part, with information handling system 146. Information handling system 146 may comprise any instrumentality or aggregate of instrumentalities operable to compute, estimate, classify, process, transmit, receive, retrieve, originate, switch, store, display, manifest, detect, record, reproduce, handle, or utilize any form of information, intelligence, or data for business, scientific, control, or other purposes. For example, an information handling system 146 may be a processing unit 150, a network storage device, or any other suitable device and may vary in size, shape, performance, functionality, and price. Information handling system 146 may comprise random access memory (RAM), one or more processing resources such as a central processing unit (CPU) or hardware or software control logic, ROM, and/or other types of nonvolatile memory. Additional components of the information handling system 146 may comprise one or more disk drives, one or more network ports for communication with external devices as well as an input device 152 (e.g., keyboard, mouse, etc.) and video display 154. Information handling system 146 may also comprise one or more buses operable to transmit communications between the various hardware components.

Alternatively, systems and methods of the present disclosure may be implemented, at least in part, with non-transitory computer-readable media 156. Non-transitory computer-readable media 156 may comprise any instrumentality or aggregation of instrumentalities that may retain data and/or instructions for a period of time. Non-transitory computer-readable media 156 may comprise, for example, storage media such as a direct access storage device (e.g., a hard disk drive or floppy disk drive), a sequential access storage device (e.g., a tape disk drive), compact disk, CD-ROM, DVD, RAM, ROM, electrically erasable programmable read-only memory (EEPROM), and/or flash memory; as well as communications media such as wires, optical fibers, microwaves, radio waves, and other electromagnetic and/or optical carriers; and/or any combination of the foregoing.

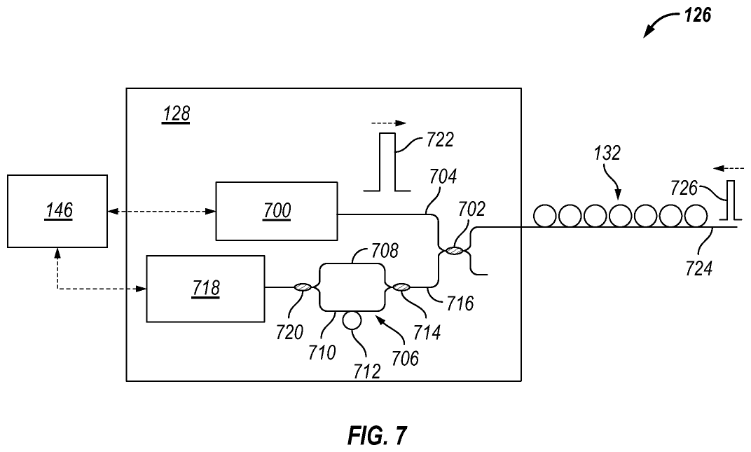

Production operations in a subsea environment may present optical challenges for a DAS based FOS system 126. For example, in a DAS system, a maximum pulse power that may be used is approximately inversely proportional to fiber length due to optical non-linearities in the fiber. Therefore, the quality of the overall signal is poorer with a longer fiber than a shorter fiber. This may impact any FOS system 126 that may utilize DAS, since the distal end of the downhole sensing fiber 132 may comprise an interval of interest (i.e., the reservoir) in which the downhole sensing fiber 132 may be deployed. The interval of interest may comprise wellbore 122 and formation 104. For pulsed DAS systems, in FOS system 126, such as the one exemplified in FIG. 7, an additional challenge is the drop-in signal to noise ratio (SNR) and spectral bandwidth associated with the decrease in the number of light pulses that may be launched into the fiber per second (i.e., DAS pulse repetition rate) when interrogating fibers with overall lengths exceeding 10 km. As such, utilizing DAS system in FOS system 126 in a subsea environment may have to increase the returned signal strength with given pulse power, increase the maximum pulse power that may be used for given fiber optic cable length, maintain the pulse power as high as possible as it propagates down the fiber optic cable length, and increase the number of light pulses that may be launched into the fiber optic cable per second.

FIG. 7 illustrates an example of DAS system for FOS 126. The DAS system may comprise information handling system 146 that is communicatively coupled to interrogator 128. Without limitation, DAS system may comprise a coherent Rayleigh scattering system with a compensating interferometer. In examples, the DAS system may be used for phase-sensitive sensing of events in a wellbore using measurements of coherent Rayleigh backscatter and/or may interrogate a downhole sensing fiber containing an array of partial reflectors, for example, fiber Bragg gratings.

As illustrated in FIG. 7, interrogator 128 may comprise a pulse generator 700 coupled to a first coupler 702 using an optical fiber 704. Pulse generator 700 may be a laser, or a laser connected to at least one amplitude modulator, or a laser connected to at least one switching amplifier, i.e., semiconductor optical amplifier (SOA). First coupler 702 may be a traditional fused type fiber optic splitter, a circulator, a PLC fiber optic splitter, or any other type of splitter known to those with ordinary skill in the art. Pulse generator 700 may be coupled to optical gain elements (not shown) to amplify pulses generated therefrom. Example optical gain elements comprise Erbium Doped Fiber Amplifiers (EDFAs) or Semiconductor Optical Amplifiers (SOAs).

FOS system 126, which is a DAS system, may comprise an interferometer 706. Without limitations, interferometer 706 may comprise a Mach-Zehnder interferometer. For example, a Michelson interferometer or any other type of interferometer 706 may also be used without departing from the scope of the present disclosure. Interferometer 706 may comprise a top interferometer arm 708, a bottom interferometer arm 710, and a gauge 712 positioned on bottom interferometer arm 710. Interferometer 706 may be coupled to first coupler 702 through a second coupler 714 and an optical fiber 716. Interferometer 706 further may be coupled to a photodetector assembly 718 of the DAS system through a third coupler 720 opposite second coupler 714. Second coupler 714 and third coupler 720 may be a traditional fused type fiber optic splitter, a PLC fiber optic splitter, or any other type of optical splitter known to those with ordinary skill in the art. Photodetector assembly 718 may comprise associated optics and signal processing electronics (not shown). Photodetector assembly 718 may be a semiconductor electronic device that uses the photoelectric effect to convert light to electricity. Photodetector assembly 718 may be an avalanche photodiode or a pin photodiode but is not intended to be limited to such.

When operating FOS system 126, pulse generator 700 may generate a first optical pulse 722 which is transmitted through optical fiber 704 to first coupler 702. First coupler 702 may direct first optical pulse 722 through a sensing fiber 724. It should be noted that sensing fiber 724 may be at least a part of downhole sensing fiber 132 (e.g., referring to FIGS. 1A and 1B). As illustrated, sensing fiber 724 may be coupled to first coupler 702. As first optical pulse 722 travels through sensing fiber 724, imperfections in sensing fiber 724 may cause a portion of the light to be backscattered along sensing fiber 724 due to Rayleigh scattering. In other embodiments, the sensing fiber 724 may be enhanced (or engineered) to yield a higher-than-Rayleigh backscatter coefficient. Scattered light according to Rayleigh scattering is returned from every point along sensing fiber 724 along the length of sensing fiber 724 and is shown as backscattered light 726 in FIG. 7. This backscatter effect may be referred to as Rayleigh backscatter. Density fluctuations in sensing fiber 724 may give rise to energy loss due to the scattered light, α_{scat}, with the following coefficient:

$${α}_{scat} = \frac{8 {π}^{3}}{3 {λ}^{4}} {n}^{8} {p}^{2} {kT}_{f} β \qquad ( 1 )$$

where n is the refraction index, ρ is the photoelastic coefficient of sensing fiber 724, k is the Boltzmann constant, and β is the isothermal compressibility. T_{f} is a fictive temperature, representing the temperature at which the density fluctuations are “frozen” in the material. Fiber optical cable 724 may be terminated with a low reflection device (not shown). In examples, the low reflection device (not shown) may be a fiber coiled and tightly bent to violate Snell's law of total internal reflection such that all the remaining energy is sent out of sensing fiber 724.

Backscattered light 726 may travel back through sensing fiber 724, until it reaches second coupler 714. First coupler 702 may be coupled to second coupler 714 on one side by optical fiber 716 such that backscattered light 726 may pass from first coupler 702 to second coupler 714 through optical fiber 716. Second coupler 714 may split backscattered light 726 based on the number of interferometer arms so that one portion of any backscattered light 726 passing through interferometer 706 travels through top interferometer arm 708 and another portion travels through bottom interferometer arm 710. Therefore, second coupler 714 may split the backscattered light from optical fiber 716 into a first backscattered pulse and a second backscattered pulse. The first backscattered pulse may be sent into top interferometer arm 708. The second backscattered pulse may be sent into bottom interferometer arm 710. These two portions may be re-combined at third coupler 720, after they have exited interferometer 706, to form an interferometric signal.

Interferometer 706 may facilitate the generation of the interferometric signal through the relative phase shift variations between the light pulses in top interferometer arm 708 and bottom interferometer arm 710. Specifically, gauge 712 may cause the length of bottom interferometer arm 710 to be longer than the length of top interferometer arm 708. With different lengths between the two arms of interferometer 706, the interferometric signal may comprise backscattered light from two positions along sensing fiber 724 such that a phase shift of backscattered light between the two different points along sensing fiber 724 may be identified in the interferometric signal. The distance between those points L may be half the length of the gauge 712 in the case of a Mach-Zehnder configuration, or equal to the gauge length in a Michelson interferometer configuration.

While FOS system 126 is running, the interferometric signal will typically vary over time. The variations in the interferometric signal may identify strains in sensing fiber724 that may be caused, for example, by seismic energy. By using the time of flight for first optical pulse 722, the location of the strain along sensing fiber 724 and the time at which it occurred may be determined. If sensing fiber 724 is positioned within a wellbore, the locations of the strains in sensing 724 may be correlated with depths in the formation in order to associate the seismic energy with locations in the formation and wellbore.

To facilitate the identification of strains in sensing fiber 724, the interferometric signal may reach photodetector assembly 718, where it may be converted to an electrical signal. The photodetector assembly may provide an electric signal proportional to the square of the sum of the two electric fields from the two arms of the interferometer. This signal is proportional to:

P(t)=P1+P2+2*\sqrt{(P1P2)cos(ϕ−ϕ2)} (2)
where Pₙ is the power incident to the photodetector from a particular arm (1 or 2) and ϕₙ is the phase of the light from the particular arm of the interferometer. Photodetector assembly 718 may transmit the electrical signal to information handling system 146, which may process the electrical signal to identify strains within sensing fiber 724 and/or convey the data to a display and/or store it in computer-readable media. Photodetector assembly 718 and information handling system 146 may be communicatively and/or mechanically coupled. Information handling system 146 may also be communicatively or mechanically coupled to pulse generator 700.

Modifications, additions, or omissions may be made to FIG. 7 without departing from the scope of the present disclosure. For example, FIG. 7 shows a particular configuration of components of a DAS system, which is a FOS system 126, operating via optical time-domain reflectometry (OTDR). However, any suitable configurations of components may be used, such that the DAS system may be operated via optical frequency-domain interferometry (OFDR). As another example, pulse generator 700 may generate a multitude of coherent light pulses, optical pulse 722, operating at distinct frequencies that are launched into the sensing fiber 724 either simultaneously or in a staggered fashion. For example, the photo detector assembly is expanded to feature a dedicated photodetector assembly for each light pulse frequency. In examples, a compensating interferometer may be placed in the launch path (i.e., prior to traveling down sensing fiber 724) of the interrogating pulse to generate a pair of pulses that travel down sensing fiber 724. In examples, interferometer 706 may not be necessary to interfere the backscattered light from pulses prior to being sent to photo detector assembly. In one branch of the compensation interferometer in the launch path of the interrogating pulse, an extra length of fiber not present in the other branch (a gauge length similar to gauge 712 of FIG. 7) may be used to delay one of the pulses. To accommodate phase detection of backscattered light using FOS system 126, one of the two branches may comprise an optical frequency shifter (for example, an acousto-optic modulator) to shift the optical frequency of one of the pulses, while the other may comprise a gauge. This may allow using a single photodetector receiving the backscatter light to determine the relative phase of the backscatter light between two locations by examining the heterodyne beat signal received from the mixing of the light from different optical frequencies of the two interrogation pulses.

In examples, the DAS system, which is a FOS system 126, may generate interferometric signals for analysis by the information handling system 146 without the use of a physical interferometer. For instance, the DAS system may direct backscattered light to photodetector assembly 718 without first passing it through any interferometer, such as interferometer 706 of FIG. 7. Alternatively, the backscattered light from the interrogation pulse may be mixed with the light from the laser originally providing the interrogation pulse. Thus, the light from the laser, the interrogation pulse, and the backscattered signal may all be collected by photodetector assembly 718 and then analyzed by information handling system 146. The light from each of these sources may be at the same optical frequency in a homodyne phase demodulation system or may be different optical frequencies in a heterodyne phase demodulator. This method of mixing the backscattered light with a local oscillator allows measuring the phase of the backscattered light along the fiber relative to a reference light source.

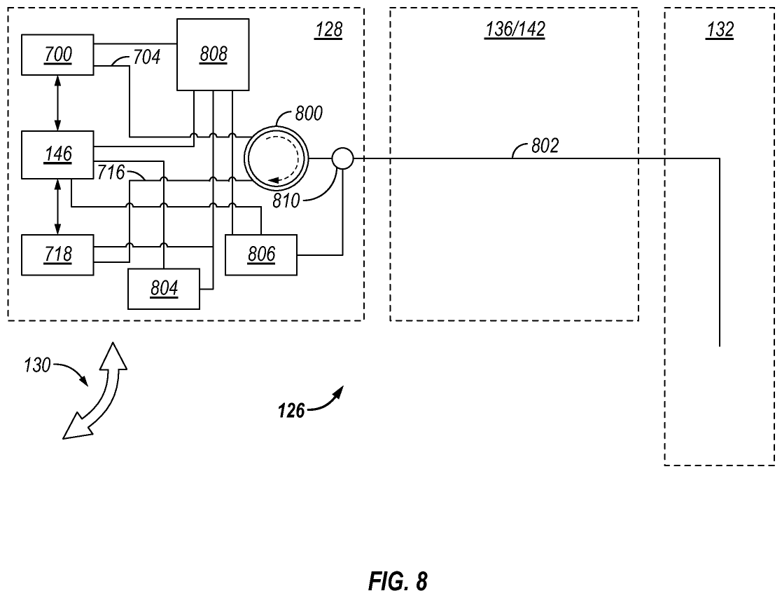

FIG. 8 illustrates an example of DAS system, which is a FOS system 126, which may be designed for deployment in a subsea environment. As illustrated, FOS system 126 may comprise interrogator 128, a static umbilical line 136 or optical flying lead 142, and downhole sensing fiber 132. It should be noted that there may be a plurality of downhole sensing fibers 132 disposed in wellbore 122. Additionally, downhole sensing fibers 132 may have one or more measurement instruments attached to each of some of the downhole sensing fibers 132. Interrogator 128 may be marinized to withstand deep sea pressure and corrosive nature of the sea and sea floor 106 as interrogator 128 is disposed on sea floor 106 during operations. For example, interrogator 128 may be housed within an atmospheric pressure chamber with or without temperature control to keep delicate electro-optical circuits dry and free of conductive sea water. Deep sea temperatures may approach close to the freezing point of water near OC (32F). This atmospheric chamber may be structurally designed to withstand pressures for up to and about 13 kpsi for seawater depths to 30,000 ft (9,500 meters). This assumes a seawater pressure gradient of about 0.432 psi/foot of vertical depth. However, designs may only be used at water depths of about 10,000 ft (3,000 meters) which is equivalent to about 4,320 psi. If temperature-sensitive optical components, such as laser oscillators may be employed, then active temperature stabilization may be utilized, via resistive heater elements, thermoelectric devices, and/or use of material insulation or vacuum within the chamber to reduce fast temperature variation when lowering the protected instrument to the sea floor for limited mission profiles. For longer term mission profiles, controlled active heating of the marinized atmospheric chamber may be utilized.

As illustrated, interrogator 128 may comprise a pulse generator 700 and photodetector assembly 718, both of which may be communicatively coupled to an information handling system 146. Pulse generator 700 and photodetector assembly 718 may operate and function as described above. In examples, pulse generator 700 may be any form of ultra-fast optical switch or optical modulator, such as a semiconductor optical amplifier (SOA) or electro-optical modulator (EOM) or acousto-optic modulator (AOM) or magneto-optic modulator (MOM), which effectively gates the optical intensity or power or acts to modulate the phase of the traversing optical energy travelling through said pulse generator. Information handling system 146 may control the operation of pulse generator 700 and photodetector assembly 718. For example, information handling system 146 may control when pulse generator 700 activates and transmits light pulses.

The transmitted light pulses from pulse generator 700 may enter optical fiber 704, which may attach pulse generator 700, and traverse optical fiber 704 to a circulator 800. Circulator 800 may connect pulse generator 700 to a fiber optic cable 802. Second fiber optic cable may be disposed in static umbilical line 136 or optical flying lead 142. While illustrated as a single fiber optic cable, fiber optic cable 802 may be a plurality of fused fibers fused or connected together to form a single fiber optic cable. In examples, circulator 800 functions to steer light unidirectionally between one or more input and outputs of circulator 800. Without limitation, circulators 800 are passive three-port devices wherein light from a first port is split internally into two independent polarization states and wherein these two polarization states are made to propagate two different paths inside circulator 800. These two independent paths allow one or both independent light beams to be rotated in polarization state via the Faraday effect in optical media. Polarization rotation of the light propagating through free space optical elements within the circulator thus allows the total optical power of the two independent beams to uniquely emerge together with the same phase relationship from a second port of circulator 800.

Conversely, if any light enters the second port of circulator 800 in the reverse direction, the internal free space optical elements within circulator 800 may operate identically on the reverse direction light to split it into two polarizations states. After appropriate rotation of polarization states, these reverse in direction polarized light beams, are recombined, as in the forward propagation case, and emerge uniquely from a third port of circulator 800 with the same phase relationship and optical power as they had before entering circulator 800. Additionally, circulator 800 may act as a gateway, which may only allow chosen wavelengths of light to pass through circulator 800 and pass to fiber optic cable 802, which is connected to downhole sensing fiber 132.

Light may reflect off the end of downhole sensing fiber 132 as backscatter light 726 (e.g., referring to FIG. 7). Backscatter light 726 may travers back through downhole sensing fiber 132 and into static umbilical line 136 or optical flying lead 142 to arrive at circulator 800. Circulator 800 may further be connected to photodetector assembly 718. Circulator 800 may guide the light through optical fiber 716 to photodetector assembly 718. The sensed backscatter light 726 may be stored as data and transmitted to information handling system 146 for storage.

With continued reference to FIG. 8, energy source 808 may power pulse generator 700, photodetector assembly 718, information handling system 146, transmitter 804, and timekeeper 806. Energy source 808 may be batteries, for example. All electrical energy sources and other electro-optical components must be able to operate at temperatures at or about OC (32F).

During measurement operations, timekeeper 806 may be utilized to control when light is emitted from pulse generator 700. Timekeeper 806 may be a quartz clock, atomic clock or GPS timing signal telemetered, via said acoustic or optical telemetry systems, from a GPS receiver on surface or floating above the marinized subsea system. As illustrated in FIG. 8, timekeeper 806 may also be communicatively coupled to a fiber stretcher 810 which is placed in-line with fiber optic cable 802. Fiber stretcher 810 may be constructed of a piezoelectric crystalline transducer (PZT), which may be shaped as a smooth cylinder or ring and made of lead-zirconate-titanate, for example, with optical fiber(s) wound around the cylindrical surface of the crystal. Optical fibers that may be tightly wound and bonded to the PZT surface may effectively be made to strain or stretch statically and/or dynamically, via “hoop mode” electro-mechanical crystal transduction, by an applied voltage potential across said PZT, which has at least one-pair of thin conductive metal electrodes between the inner and outer surfaces of the crystal itself. Metal electrodes may allow for an electric field to be passed across the dielectric crystal material which results in electrostriction displacement effects to change the crystals radial thickness, thus imparting a change in length along the optical fiber length directly wound onto the crystal. Note that other mechanical transduction modes of the PZT may be utilized to stretch (strain) the optical fiber. Fiber stretcher 810 may allow for timekeeper 806 to encode a global positioning system (GPS) time signal onto backscatter light 726 following inter-range instrumentation group (TRIG) timecode standard using a carrier frequency that is below half the pulse repetition rate used to acquire the measurement data (i.e., a Nyquist frequency). Inter-range instrumentation group timecodes, commonly known as TRIG timecode, are standard formats for transferring timing information. Atomic frequency standards and GPS receivers designed for precision timing are often equipped with an TRIG output. The standards were created by the Tele Communications Working Group of the U.S. military's Inter-Range Instrumentation Group (IRIG), the standards body of the Range Commanders Council. Work on these standards started in October 1956, and the original standards were accepted in 1960. In addition to or instead of the TRIG timecode signal (which encodes the absolute GPS time), a GPS time-synchronized pulse per second (PPS) may be encoded onto backscatter light 726 where the rising or falling edge of the PPS repeats exactly once per second with up to nano-second precision. Timekeeper 806 is connected to and controlled by information handling system 146, which communicates operations and timing of the operations to personnel on floating vessel 102 (e.g., referring to FIG. 1) using transmitter 804.

FIG. 8 further illustrates a transmitter 804 that is connected to information handling system 146. Transmitter 804 may communicate measurement data from photodetector assembly 718 as well as operational information. Operational information may comprise activating pulse generator 700. In examples, transmitter 804 may be a radio frequency (RF) transmitter, an acoustic transmitter, an optical transmitter, and/or an electrical transmitter. As illustrated in FIG. 8 and FIG. 1, wireless communication 130 may be wireless and operate at an ultra-low frequency of 10 kHz or less. Wireless communication 130 may comprise data packets transmitted from floating vessel 102 to interrogator 128. Data packets may comprise instructions for operations, prevent drift of timekeeper 806, subsea instrumentation health, and/or remaining battery charge level and capacity. For example, during operations, personnel may specify a desired time to start measurement operations. The desired start time is communicated to interrogator 128, specifically information handling system 146 disposed in interrogator 128, through wireless communication 130 and transmitter 804. Information handling system 146 may activate pulse generator 700 to start pulsing at the rising edge of the PPS corresponding to the desired start time, which may be controlled by timekeeper 806. This ensures that the first data samples that are stored to disk and/or streamed to the drill ship/platform precisely matches the personnel-specified start time. These recorded timing signals may be used for synchronization of the DAS acoustic source boat operations during active or passive seismic operations.

Systems and methods described functionally provide an all-optical downhole sensing solution for subsea wells, enabling the simultaneous measurements of temperature, pressure, acoustics, and/or strain in downhole sensing fibers. The system and methods described are inherently compliant with the Intelligent Well Interface Standardization (IWIS) and SEAFOM recommended practices. Systems and methods described functionally provide an all-optical downhole sensing solution for subsea wells. In practice, the systems and methods may minimize the number of transmission fibers providing optical continuity from topside to optical flying lead, thus saving significant complexity and costs in subsea optical infrastructure and installation thereof. Additionally, systems and methods described above may maximize the number of downhole sensing fibers that may be configured for any combination of fiber optic sensing applications. In particular, the systems and methods can enable simultaneous DAS, DSS, DTS, and FBG sensing of subsea completions.

Improvements over current technology may be found in the methods and systems described above. Specifically, improvements may be found in temporary remotely record DAS, DTS and DSS signal data without physical umbilical connectivity to a surface station such an onshore or offshore platform or waiting/permanent floating vessel or buoy. This may effectively reduce operations costs for monitoring in well fiber optic distributed sensing systems over the production lifetime of the well.

The systems and methods for a fiber optic sensing system discussed above, implemented within a subsea environment may comprise any of the various features of the systems and methods disclosed herein, including one or more of the following statements.

Statement 1: A fiber optic sensing (FOS) system may comprise an interrogator that is marinized to be disposed on a sea floor, a fiber optic cable optically connected to the interrogator, and one or more downhole sensing fibers optically connected to the fiber optic cable.

Statement 2: The FOS system of statement 1, wherein the interrogator further comprises a timekeeper that is connected to the fiber optic cable through a fiber stretcher.

Statement 3: The FOS system of statement 2, wherein the timekeeper is a quartz clock.

Statement 4: The FOS system of statement 2, wherein the timekeeper is configured to encode a global positioning system (GPS) time signal onto a light pulse transmitted from the interrogator.

Statement 5: The FOS system of statement 4, wherein the GPS time signal is encoded using an Inter-Range Instrumentation Group (TRIG) timecode.

Statement 6: The FOS system of statement 2, wherein the timekeeper is configured to encode a global positioning system (GPS) time-synchronized pulse per second (PPS) onto a light pulse transmitted from the interrogator.

Statement 7: The FOS system of any previous statements 1 or 2, wherein the fiber optic cable is disposed in an optical flying lead or a static umbilical line.

Statement 8: The FOS system of any previous statements 1, 2, or 7, wherein the interrogator may further comprise an atmospheric pressure chamber, an energy source disposed in the atmospheric pressure chamber, a pulse generator disposed in the atmospheric pressure chamber and connected to the energy source and an information handling system, a photodetector assembly disposed in the atmospheric pressure chamber and connected to the energy source and the information handling system, and a transmitter disposed in the atmospheric pressure chamber and connected to the information handling system.

Statement 9: The FOS system of statement 8, wherein the energy source is a battery.

Statement 10: The FOS system of statement 8, wherein the transmitter is a radio frequency (RF) transmitter.

Statement 11: A method may comprise disposing an interrogator that is marinized on a sea floor, connecting a fiber optic cable to the interrogator, and connecting one or more downhole sensing fibers to the fiber optic cable.

Statement 12: The method of statement 11, further comprising encoding a global positioning system (GPS) time signal onto a light pulse transmitted from the interrogator with a timekeeper disposed in the interrogator.

Statement 13: The method of statement 12, wherein the timekeeper is connected to the fiber optic cable through a fiber stretcher.

Statement 14: The method of statement 13, wherein the timekeeper is a quartz clock.

Statement 15: The method of any previous statements 11 or 12, further comprising encoding a GPS time signal using an Inter-Range Instrumentation Group (TRIG) timecode onto a light pulse transmitted from the interrogator with a timekeeper disposed in the interrogator.

Statement 16: The method of any previous statements 11, 12, or 15, further comprising encoding a global positioning system (GPS) time-synchronized pulse per second (PPS) onto a light pulse transmitted from the interrogator with a timekeeper disposed in the interrogator.

Statement 17: The method of any previous statements 11, 12, 15, or 16, wherein the fiber optic cable is disposed in an optical flying lead or a static umbilical line.

Statement 18: The method of any previous statements 11, 12, 15, 16, or 17, wherein the interrogator may further comprise an atmospheric pressure chamber, an energy source disposed in the atmospheric pressure chamber, a pulse generator disposed in the atmospheric pressure chamber and connected to the energy source and an information handling system, a photodetector assembly disposed in the atmospheric pressure chamber and connected to the energy source and the information handling system, and a transmitter disposed in the atmospheric pressure chamber and connected to the information handling system.

Statement 19: The method of statement 18, wherein the energy source is a battery.

Statement 20: The method of statement 18, wherein the transmitter is a radio frequency (RF) transmitter.

Although the present disclosure and its advantages have been described in detail, it should be understood that various changes, substitutions and alterations may be made herein without departing from the spirit and scope of the disclosure as defined by the appended claims. The preceding description provides various examples of the systems and methods of use disclosed herein which may contain different method steps and alternative combinations of components. It should be understood that, although individual examples may be discussed herein, the present disclosure covers all combinations of the disclosed examples, including the different component combinations, method step combinations, and properties of the system. It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any comprised range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present examples are well adapted to attain the ends and advantages mentioned as well as those that are inherent therein. The particular examples disclosed above are illustrative only and may be modified and practiced in different but equivalent manners apparent to those skilled in the art having the benefit of the teachings herein. Although individual examples are discussed, the disclosure covers all combinations of all of the examples. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative examples disclosed above may be altered or modified and all such variations are considered within the scope and spirit of those examples. If there is any conflict in the usages of a word or term in this specification and one or more patent(s) or other documents that may be incorporated herein by reference, the definitions that are consistent with this specification should be adopted.

What is claimed is:

1. A fiber optic sensing (FOS) system comprising:
an interrogator that is marinized to be disposed on a sea floor;
a fiber optic cable optically connected to the interrogator; and
one or more downhole sensing fibers optically connected to the fiber optic cable.

2. The FOS system of claim 1, wherein the interrogator further comprises a timekeeper that is connected to the fiber optic cable through a fiber stretcher.

3. The FOS system of claim 2, wherein the timekeeper is a quartz clock.

4. The FOS system of claim 2, wherein the timekeeper is configured to encode a global positioning system (GPS) time signal onto a light pulse transmitted from the interrogator.

5. The FOS system of claim 4, wherein the GPS time signal is encoded using an Inter-Range Instrumentation Group (TRIG) timecode.

6. The FOS system of claim 2, wherein the timekeeper is configured to encode a global positioning system (GPS) time-synchronized pulse per second (PPS) onto a light pulse transmitted from the interrogator.

7. The FOS system of claim 1, wherein the fiber optic cable is disposed in an optical flying lead or a static umbilical line.

8. The FOS system of claim 1, wherein the interrogator further comprises:
an atmospheric pressure chamber;
an energy source disposed in the atmospheric pressure chamber;
a pulse generator disposed in the atmospheric pressure chamber and connected to the energy source and an information handling system;
a photodetector assembly disposed in the atmospheric pressure chamber and connected to the energy source and the information handling system; and
a transmitter disposed in the atmospheric pressure chamber and connected to the information handling system.

9. The FOS system of claim 8, wherein the energy source is a battery.

10. The FOS system of claim 8, wherein the transmitter is a radio frequency (RF) transmitter.

11. A method comprising:
disposing an interrogator that is marinized on a sea floor;
connecting a fiber optic cable to the interrogator; and
connecting one or more downhole sensing fibers to the fiber optic cable.

12. The method of claim 11, further comprising encoding a global positioning system (GPS) time signal onto a light pulse transmitted from the interrogator with a timekeeper disposed in the interrogator.

13. The method of claim 12, wherein the timekeeper is connected to the fiber optic cable through a fiber stretcher.

14. The method of claim 13, wherein the timekeeper is a quartz clock.

15. The method of claim 11, further comprising encoding a GPS time signal using an Inter-Range Instrumentation Group (TRIG) timecode onto a light pulse transmitted from the interrogator with a timekeeper disposed in the interrogator.

16. The method of claim 11, further comprising encoding a global positioning system (GPS) time-synchronized pulse per second (PPS) onto a light pulse transmitted from the interrogator with a timekeeper disposed in the interrogator.

17. The method of claim 11, wherein the fiber optic cable is disposed in an optical flying lead or a static umbilical line.

18. The method of claim 11, wherein the interrogator further comprises:
an atmospheric pressure chamber;
an energy source disposed in the atmospheric pressure chamber;
a pulse generator disposed in the atmospheric pressure chamber and connected to the energy source and an information handling system;
a photodetector assembly disposed in the atmospheric pressure chamber and connected to the energy source and the information handling system; and
a transmitter disposed in the atmospheric pressure chamber and connected to the information handling system.

19. The method of claim 18, wherein the energy source is a battery.

20. The method of claim 18, wherein the transmitter is a radio frequency (RF) transmitter.
